(19) United States
(12) Patent Application Publication
(10) Pub. No.: US 2010/0249624 A1
(43) Pub. Date: Sep. 30, 2010

(54) DEVICE FOR ANALYZING QUALITY OF SLEEP

(75) Inventors: Yung-Kang PENG, Taipei City (TW); Wei-Chao Peng, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: PLATINUM TEAM CO., LTD., Taipei City (TW)

(21) Appl. No.: 12/731,506

(22) Filed: Mar. 25, 2010

(30) Foreign Application Priority Data
Mar. 27, 2009 (TW) 098204898

(51) Int. Cl. A61B 5/04

(52) U.S. Cl. 600/509

(57) ABSTRACT

The present invention relates to a device for analyzing quality of sleep, which contains multiple electrode patches attached to human body and an electro-cardio measurement unit connected with the electrode patches. The device of the present invention provides an electro-cardio signal by the multiple electrode patches attached to human body and analyzes the electro-cardio signal by an analyzing unit to know the quality of sleep.

### FIELD OF THE INVENTION

The present invention relates to a kind of analyzing device, especially an analyzing device to analyze quality of sleep.

### BACKGROUND OF THE INVENTION

In accordance with the concept of preventive medicine, people have begun establishing the habits of periodic health examination and have valued the trouble sleeping that takes ⅓ of sleeping time gradually. According to surveys, more than half of us have suffered anyone of sleeping disorders. Hospitals start to set up sleeping center sequentially to aim at more than million groups of trouble sleeping, however, it's nothing more than great suffering for modern people who care about the efficiency to go through the treatment, inspection, and receiving the report, which spends months of time.

Furthermore, the electro-cardio signal generated by human body relates to all kinds of healthy condition. Because we are able to understand the working function of whole body through the signal, several kinds of device which used to measure the signal were developed. For example, the device for medical use or general use of understanding the sleep condition makes users understand their body condition by the measured electro-cardio signal.

In a general electro-cardio measurement device, which uses electrode patches, wires and a computer device to carry out measurement, meaning that attaching the electrode patches to a human body and making connection with wires between electrode patches and the computer device, are able to transfer the electro-cardio signal detected by the electrode patches, and send the signal to the computer device. Subsequently, the computer device will save, analyze or show the received electro-cardio signal.

Refer to the above electro-cardio measurement device, the traditional device on the market or for medical use of detecting the sleep condition are too complex and heavy, which let patients difficult to sleep because of the unfamiliar environment (sleep lab), the complex signal lines attached to the body, and the technicians inspecting all night.

Therefore, aiming at these issues to address a novel device of sleep quality analysis device is a home-based device. The device of the present invention is easy to operate and only need few minutes to install by oneself and the patients are allowed to sleep on their own bed. Accordingly, the present invention provides a low-interference and fast way of sleep inspection.

### BRIEF DESCRIPTION OF DRAWINGS

FIG. 1 shows a block diagram of a preferred embodiment of the present invention.

FIG. 2 shows a block diagram of another preferred embodiment of the present invention.

FIG. 3 shows a stereogram of a preferred embodiment of the present invention.

FIG. 4 shows a stereogram of another preferred embodiment of the present invention.

FIG. 5 shows a stereogram of another preferred embodiment of the present invention in a different perspective of FIG. 4.

The illustration of the main components:
10: electrode patch
12: electrode patch
14: electro-cardio measurement unit
140: amplification circuit
142: digital-to-analog converting unit
16: analyzing unit
18: processing unit
20: recording unit
22: transmission interface
24: display unit
26: power supply unit
30: shell
32: button groove
40: shell
42: switch
44: button groove

### SUMMARY OF THE INVENTION

The present invention provides a device for analyzing quality of sleep, which contains multiple electrode patches attached to a human body, an electro-cardio measurement unit connected with the electrode patches to produce an electro-cardio signal by measuring the human body through the electrode patches, and an analyzing unit to analyze the electro-cardio signal and know the quality of sleep.

### DETAILED DESCRIPTION OF THE INVENTON

The present invention provides a device for analyzing quality of sleep, which detects the quality of sleep by an electro-cardio signal of human body analyzed by an analyzing unit.

The device of the present invention is convenient to users to wear by removing complex wires.

The device of the present invention is convenient to be taken off and replaced by multiple button grooves.

The device of the present invention obtains users' electro-cardio signals by wireless detection.

The device of the present invention contains multiple electrode patches attached to a human body, an electro-cardio measurement unit connected with the electrode patches to produce an electro-cardio signal by measuring the human body through the electrode patches, and an analyzing unit to analyze the electro-cardio signal and know the quality of sleep.

The device of the present invention contains multiple electrode patches attached to a human body, an electro-cardio measurement unit connected with the electrode patches to produce an electro-cardio signal by measuring the human body through the electrode patches, and an analyzing unit to analyze the electro-cardio signal and know the quality of sleep.

The device of the present invention further contains a transmission interface coupled with a transmission unit to transmit the electro-cardio signal to the analyzing unit, wherein the transmission interface is wired or wireless, or is Internet.

The device of the present invention is able to detect the body position, such as supine, lying, left side, or right side position, and decide the signal whether normal or not within one minute after starting recording the signal.

### Example

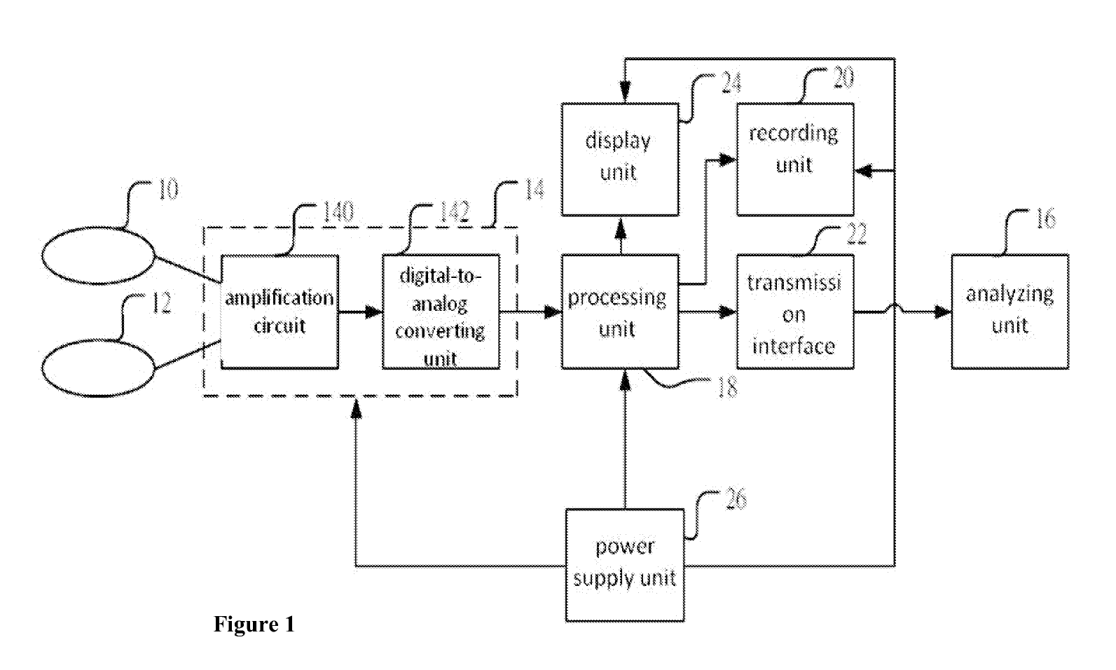

As shown in FIG. 1, one of the preferred embodiment of the present invention, the device contained multiple electrode patches 10, 12, an electro-cardio measurement unit 14, and an analyzing unit 16. The device contained at least 2 electrode patches 10, 12, which were able to attach to the skin of abdomen or nearby the heart for detecting the electro-cardio signal conveniently, and was convenient to users to wear by removing complex wires. The electro-cardio measurement unit 14 was connected with the electrode patched 10, 12, and measured human body through the electrode patched 10, 12 to produce an electro-cardio signal, and the analyzing unit 16 analyzes the electro-cardio signal to know the quality of sleep.

In an embodiment of the device of the present invention, wherein the electro-cardio measurement unit 14 contained an amplification circuit 140 used to amplify the electro-cardio signal of human body, and a digital-to-analog converting unit 142 used to convert the amplified electro-cardio signal to a digital signal for analyzing by the analyzing unit 16.

Bearing on the above, the analyzing unit 16 analyzed the electro-cardio signal by a cardiopulmonary coupling to know the quality of sleep by analyzing the electro-cardio signal, wherein the cardiopulmonary coupling which provided a more convenient way of sleep inspection was the sleep cardiopulmonary coupling mapping analysis based on electrocardiography, the accuracy was almost 90 percents, compared with home inspection and the polysomnography, standard of the sleep medical community. Furthermore, contrasted to the polysomnography, which let patients difficult to sleep because of the unfamiliar environment (sleep lab), the complex signal lines attached to the body, and the technicians inspecting all night, the device of the present was easy to operate and only need few minutes to install by oneself. The patients was allowed to sleep on their own bed, and handed in the device next day to get the result. The embodiment was a low-interference and fast way of sleep inspection.

The device of the present invention was a sleep inspection based on the traditional cardiopulmonary, employed complex computing analysis of cardiopulmonary coupling, and cooperated with subjects' own feelings. This cardiopulmonary sleep inspection provided an objective view of stability of sleep, and made us know the whole-night distribution of the unstable sleep to screen the Sleep Apnea.

In an embodiment of the present invention, the analyzing unit analyzed the electro-cardio signal in accordance with Hilbert-Huang Transformation (HHT), and known the quality of sleep. In a preferred embodiment, the analyzing unit 16 is a computer.

As shown in FIG. 1, the device for analyzing quality of sleep of the present invention contained a processing unit 18, a record unit 20, a transmission interface 22, a display unit 24, and a power unit 26. The processing unit 18 was used to transfer the electro-cardio signal. The recording unit 20 was used to record the electro-cardio signal measured by an electro-cardio measurement unit 14. The recording unit 20 was used to record the electro-cardio signal when subjects fell asleep, and transferred the electro-cardio signal saved by recording unit 20 to the analyzing unit 16 to analyze electro-cardio signal when subjects awake. In a preferred embodiment, the recording unit 20 was a memory card.

The transmission interface was used to transfer the electro-cardio signal to analyzing unit 16 to analyze electro-cardio signal. In a preferred embodiment, the transmission was a wired transmission interface or a wireless transmission interface, wherein the wired interface was a Universal Serial Bus (USB) or RS232. The wireless interface is Bluetooth, infrared or radio frequency identification (RFID). Furthermore, the transmission interface 22 was internet, and the analyzing unit 16 of the present invention obtained the electro-cardio signal of users by wired or wireless way.

In an embodiment of the present invention, display unit 24 coupled with processing unit 18, which was used to show the quality of sleep, electro-cardio signal, a time and a date, and the power supply unit 26, which was used to supply power to electro-cardio power measurement unit 14, processing unit 18, memory unit 20, transmission interface 22 and display unit 24. In a preferred embodiment, the power unit 26 was a rechargeable battery, such as lithium batteries or solar cells.

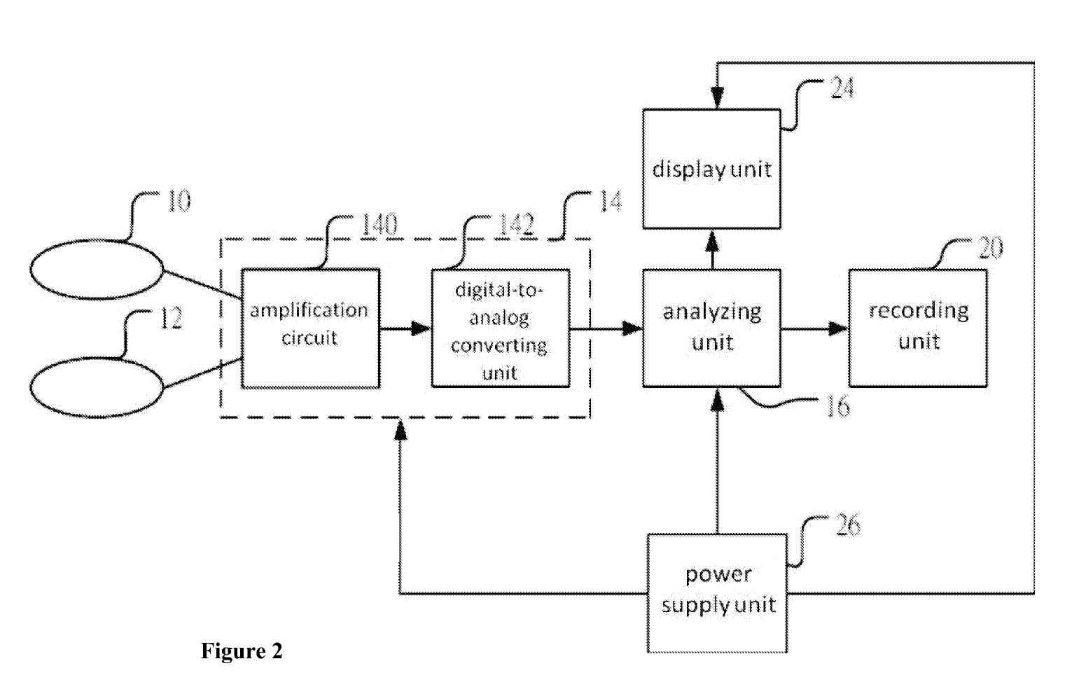

Another one of the preferred embodiments of the present invention was shown in FIG. 2. The difference between this embodiment and the embodiment shown in FIG. 1 was the fact that the analyzing unit 16 of this embodiment built in a device for analyzing quality of sleep. This embodiment was able to transfer the analyzed results of the sleep quality of the subjects to the display unit 24, and was able to use colors red, yellow and green as the expression of assessment of analyzed results to show the sleep quality. Although the present invention was incapable to replace the polysomnography, the features of the present invention, the cardiopulmonary sleep inspection, was home-based, convenient and quick-operated, which was able to benefit the sleep disorder group to reach prevention and the effect of early intervention. The device of sleep quality analysis of the present invention provided a fast way of using by built-in analysis unit 16.

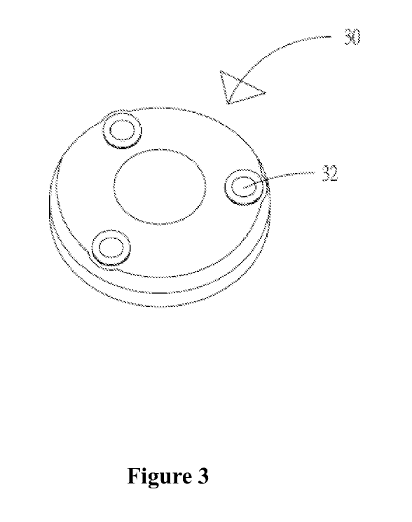

Another ne of the preferred embodiments of the present invention was shown in FIG. 3. The device of the present invention contained a shell 30 and multiple button grooves 32. Shell 30 loaded electro-cardio measurement unit 14, analyzing unit 16, processing unit 18, recording unit 20, transmission interface 22, and power supply unit 26. When fouling or damaged, the button grooves 32 on the shell 30 surface were used to fix the electrode patches, wherein dirty or damaged electrode patches were able to be removed individually to clean or replace with a new one by users.

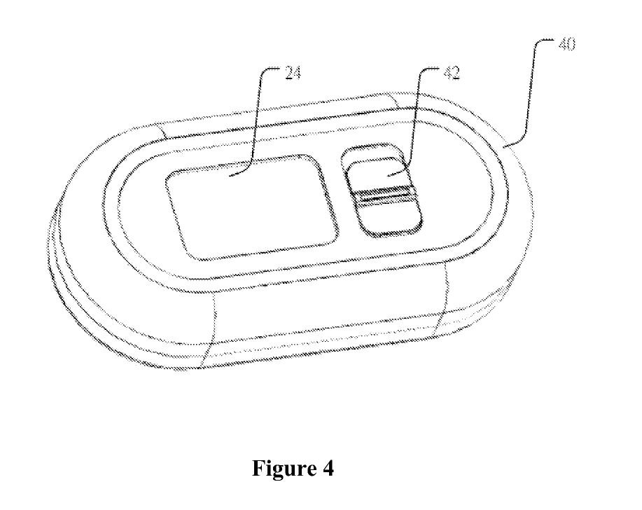

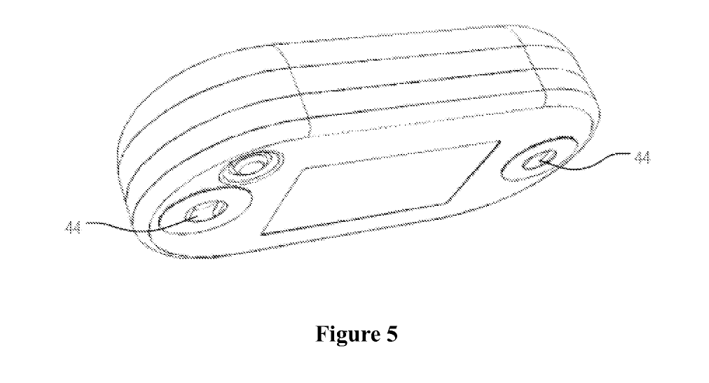

FIG. 4 and FIG. 5 showed another one of the preferred embodiments of the present invention in different perspective. The difference between this embodiment and the prior embodiment shown in FIG. 3 was that the shape of the shell 40 in this embodiment was an ellipse or a circle according to users' need. The shell 40 of this embodiment contained the display unit 24, a switch 42 and multiple button grooves 44. The display unit 24 and switch 42 were fixed on the surface of shell 40, wherein the switch 42 was used to start the proceeding measurement, and the display unit 24 was used to show the quality of sleep, electro-cardio signal, time and date. The button grooves 44 were placed under the surface of shell 40 to fix the electrode patches. The number of the button grooves and the shape of the shell were not limited to the above preferred embodiment, and it was convenient to process measurement according to users' need to design the different the number of the button grooves and the shape of the shell.

Bearing on the above, the device of the present invention used the multiple electrode patches attached to a human body and an electro-cardio measurement unit connected with the electrode patches to produce an electro-cardio signal by measuring the human body through the electrode patches, and an analyzing unit to analyze the electro-cardio signal and know the quality of sleep, wherein the electrode patch, in a preferred embodiment, was a conductive fabric, conductive mat, or conductive silicone film.

What is claimed is:

1. A device for analyzing quality of sleep, which contains multiple electrode patches attached to a human body, an electro-cardio measurement unit connected with the electrode patches to produce an electro-cardio signal by measuring the human body through the electrode patches, and an analyzing unit to analyze the electro-cardio signal and know the quality of sleep.

2. The device of claim 1, wherein the analyzing unit analyzes the electro-cardio signal by a cardiopulmonary coupling.

3. The device of claim 1, wherein the analyzing unit analyzes the electro-cardio signal in accordance with Hilbert-Huang Transformation (HHT).

4. The device of claim 1, which further contains a recording unit used to record the electro-cardio signal measuring by the electro-cardio measurement unit.

5. The device of claim 4, which further contains a processing unit used to transmit the electro-cardio signal to the recording unit for recording the electro-cardio signal.

6. The device of claim 4, wherein the recording unit is a memory card.

7. The device of claim 1, which further contains a processing unit used to transmit the electro-cardio signal to the analyzing unit.

8. The device of claim 7, which further contains a transmission interface coupled with the processing unit to transmit the electro-cardio signal to the analyzing unit.

9. The device of claim 8, wherein the transmission interface is a wired interface or a wireless interface.

10. The device of claim 9, wherein the wireless interface is Bluetooth, infrared or radio frequency identification (RFID).

11. The device of claim 9, wherein the wired interface is Universal Serial Bus (USB) or RS232.

12. The device of claim 8, wherein the interface is Internet.

13. The device of claim 7, which further contains a display unit coupled with the processing unit to display the quality of sleep, the electro-cardio signal, time, and date.

14. The device of claim 1, which further contains a power supply unit to supply power to the electro-cardio measurement unit and the analyzing unit.

15. The device of claim 1, wherein the power supply unit is a rechargeable battery.

16. The device of claim 1, wherein the electrode patch is a conductive fabric, conductive mat, or conductive silicone film.

17. The device of claim 1, which further contains a shell to load the electro-cardio measurement unit and the analyzing unit.

18. The device of claim 17, wherein the shell contains multiple button grooves on its surface to fix the electrode patches.

19. The device of claim 1, wherein the analyzing unit is a computer.

20. The device of claim 1, wherein the electro-cardio measurement unit contains an amplification circuit used to amplify the electro-cardio signal of human body, and a digital-to-analog converting unit used to convert the amplified electro-cardio signal to a digital signal for analyzing by the analyzing unit.
